(19) United States
(12) Patent Application Publication
(10) Pub. No.: US 2010/0262056 A1
(43) Pub. Date: Oct. 14, 2010

(54) BACK BRACE

(75) Inventors: Steven A. Lusky, Birdsboro, PA (US); Troy Q. Marshall, Blooming Glen, PA (US)

Correspondence Address: Saul Ewing LLP (Philadelphia);Attn: Patent Docket Clerk, Penn National Insurance Plaza, 2 North Second St., 7th Floor, Harrisburg, PA, 17101, US

(73) Assignee: X-Back Bracing Services, Inc., Birdsboro, PA (US)

(21) Appl. No.: 12/421,078

(22) Filed: Apr. 9, 2009

(51) Int. Cl. A61F 5/00

(52) U.S. Cl. 602/19; 602/36

(57) ABSTRACT

The invention is a back brace including a back panel, a front panel, and at least one dynamic tension strap for connecting the front panel to the back panel.

### FIELD OF THE INVENTION

The invention pertains to back braces. More particularly, the invention pertains to back braces for creating lift and support of the spine and abdomen.

### BACKGROUND OF THE INVENTION

There are many medical conditions, including both post-surgical and non-post-surgical conditions, in which it is necessary for a patient to wear a back brace to assist creating lift and support of the spine. Such conditions include compression fractures, herniated discs, bulging discs, severe degenerative disc diseases that cause kyphosis, chronic and acute strain associated with lumbar instability, post-surgical kyphoplasty, mechanical back pain, scoliosis, spondylosis, spondylolisthesis, and post-surgical stabilization.

Conventional back braces utilize one or more rigid bars that run vertically and/or horizontally across the wearer's torso to which is attached a lower back bracing pad that contacts the wearer's lower back.

Patients generally find the bar or bars across their torso to be uncomfortable. They also often find such back braces uncomfortable due to the lack of airflow to the portions of the body covered by the brace. Even further, conventional back braces apply pressure across the entire area of the back that they cover. However, often patients are bruised in the spinal area or may receive injections adjacent the spine which may make the area surrounding the injection site sore. Conventional back braces that apply pressure directly on the spine, therefore, may further irritate an already sore spinal area or may actually cause pain where there otherwise might be no pain by virtue of the pressure applied directly to the spine.

Conventional back braces also are generally rather complicated to fit and often require several visits to a physician in order to be correctly fitted to the patient.

Finally, conventional back braces are static in the sense that, once fitted and worn, the brace is rigid and does not adapt well to wearer movement. In fact, a common occurrence with conventional static back braces is that, as the wearer flexes and moves about while performing his or her normal activities, the back-brace often loses contact with the back. Even further, back braces tend to ride up on the torso. This has at least two undesirable effects. First, the back brace is not where it is supposed to be to properly support the desired portions of the back and hyperextend the spine. Secondly, it can be rather uncomfortable when it rides up, especially for women.

### SUMMARY OF THE INVENTION

The invention is a back brace including a back panel, a front panel, and a dynamic tensioning system comprising two dynamic tension straps attached to the back panel-that the wearer or a physician may pull forward around the wearer's torso when putting the brace on to tension the straps to achieve a desired level of compression force on the wearer's back and then attach the free ends of the two straps to the front panel of the brace to maintain that compression force.

### BRIEF DESCRIPTION OF THE DRAWINGS

FIG. 1 is a frontal view of a patient wearing a back brace in accordance with the principles of the present invention.

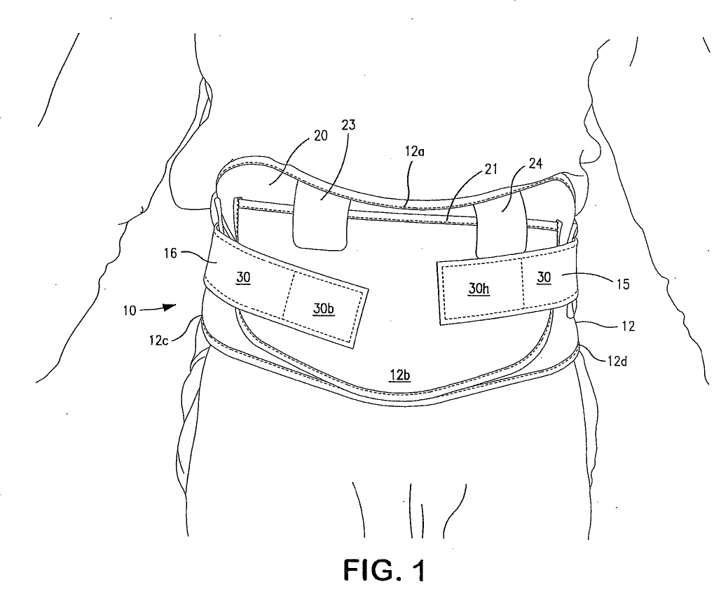

FIG. 2 is a rear view of a patient wearing a back brace in accordance with the principles of the present invention.

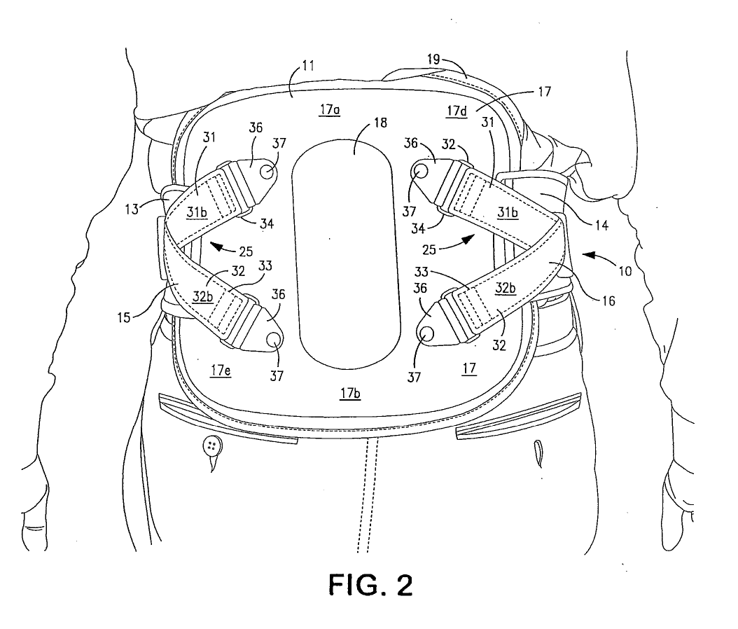

FIG. 3 is a plan view of the back brace of FIGS. 1 and 2 laid flat in preparation for being placed on a patient for fitting and/or wearing.

FIG. 4 is a pictorial of a patient adjusting the compression force-of the back brace according to one embodiment of the invention.

### DETAILED DESCRIPTION OF THE INVENTION

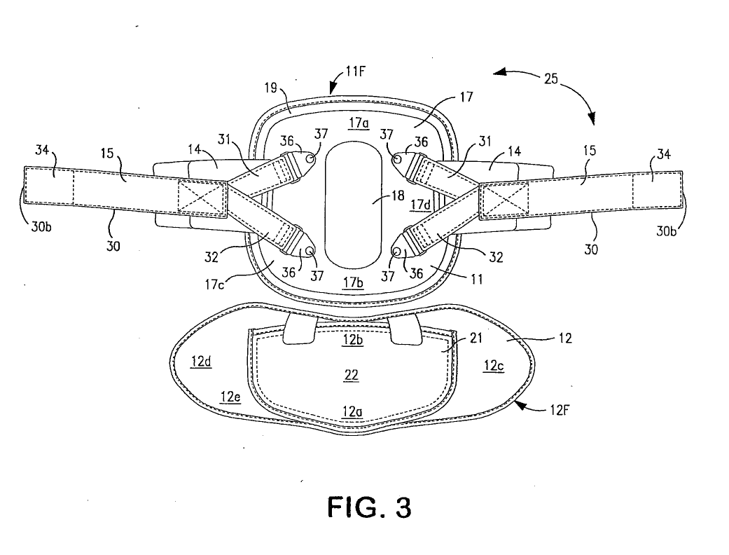

A back brace in accordance with one embodiment of the invention is a hyper-extension lumbar sacral orthotic (LSO) brace 10 providing trunk support extending from the sacral spine to the T-9 vertebrae. This is what is commonly called a standard LSO back brace. However, the invention can be incorporated into other size back braces, such as full size LSO back braces that provide trunk support extending from the sacral spine to the scapular plane or a TLSO back brace providing support from the sacral spine to the scapular plane. The back brace 10 comprises a back panel 11 for contacting the wearer's back, a front panel 12 for contacting the wearer's abdomen, two elastic straps 13, 14 (best seen in FIG. 3) for attaching the front panel 12 to the back panel 11 (as will be described in more detail below), and a dynamic force strapping system 25 comprising two additional strap assemblies 15, 16.

The use of the term “panel” with respect to front and back panels 12 and 11 is not intended to imply that they are necessarily planar in shape. In fact, in at least the illustrated embodiment, the back panel 11 is semi-rigid and has a curved shape to closely match the shape of the human back. Furthermore the front panel 12 is soft so that it will conform to the shape of whatever it is attached to, e.g., the wearer's abdomen.

The back panel 11 comprises a semi-rigid plastic piece 17, which may, for instance, be formed of 3/32″ thick ABS plastic. As used herein, the term semi-rigid means having a shape, but being elastically deformable or flexible in response to a force. The back piece 17 has a top side 17a, a bottom side 17b, a left lateral side 17c, a right lateral side, an outside major surface 17e, and an inside major surface 17f.

The above directional terms are for illustrative purposes, in relationship to the body of a person that might wear the back brace. For instance, top refers to the side towards the wearer's head, inside refers to the side facing the wearer's body, and left refers to the wearer's left. All directional terms as used herein are relative and not limiting. The semi-rigid piece 17 includes a slot (or cutout) 18 in the middle designed to correspond with the position of the spine when the brace 10 is properly worn. Back panel 11 further includes soft padding 19, such as Velfoam™ padding, on the inside surface 17f of the semi-rigid plastic piece 17 (i.e., on the side that will contact the wearer). The padding 19 can be formed of or covered with a moisture wicking material to increase wearer comfort.

The padding 19 may be attached to the piece 17 by any reasonable means, including a hook and loop attaching system, such as Velcro™, rivets, tape, adhesive, sewing, buttons, snaps, etc. In another embodiment, the padding 19 may be designed with a pocket within which the semi-rigid plastic piece 18 fits.

The slot 18 in the semi-rigid back piece 17 has the advantage of eliminating or minimizing direct pressure on the wearer's spine. This will greatly enhance comfort for many wearers insofar as many wearers will have pain or soreness at or near the spine (e.g., from the critical injury, from injections, or from a surgical incision). The semi-rigid plastic piece 17 provides virtually full support of the back area covered by the back panel 11 without applying pressure directly to the spine over most of its vertical length.

Front panel 12 has a top side 12a, bottom side 12b, left lateral side 12c, right lateral side 12d, an outside major surface 12e, and an inside major surface 12f. It also may be substantially rectangular in shape. It is formed at soft padding 20, such as ¼″ Velfoam™ of a size and shape suitable for wrapping around the wearer's abdomen. In the illustrated embodiment, the front panel 12 includes a pocket 21 for accepting another semi-rigid plastic piece 22 that can be inserted in the pocket 21 to provide rigidity to the front panel 12, if desired. In one embodiment, the plastic piece 22 (which is shown in phantom within pocket 21 in FIG. 3) also is 3/32″ ABS plastic. Two flaps 23, 24 bearing one of the hook or loop surfaces of a hook and loop attaching system may be provided for at least partially closing the opening of the pocket 21 so that the plastic piece 22 cannot fall out inadvertently, but can be readily removed upon releasing of the flaps 23, 23. The surface of the pocket comprises the other of the loop or hook surfaces.

The two elastic side straps 13,14 extend from opposite lateral sides of back panel 11 for removably attaching the front panel 12 to the back panel 11 around a wearer's torso. The straps are elastic in order to accommodate different body sizes. In addition, the straps 13, 14 are removable from either one or both of the back panel 11 and the front panel 12. For instance, in the illustrated embodiment, the straps are permanently attached to the back panel 11 by rivets 20, but are removably attached to the front panel 12 by a hook and loop attaching system. More particularly, the ends 13a, 14a of the straps 13, 14 that are to be attached to the front panel 12 are provided with one of the hook or loop surfaces of a hook and loop attaching system, such as Velcro™, and the outside surface 12e of the front panel 12 comprises the other of the hook or loop surfaces.

This feature provides two distinct advantages to the back brace 10. First, it makes it even further adjustable since the side straps 13, 14 can be attached to the front panel 12 in essentially any position of the wearer's or physician's choosing. Secondly, it makes it very easy to put on the back brace 10 insofar as one strap 13, 14 may be attached to the front panel to hold the front and back panels together loosely, the wearer can wrap the back brace around his or her torso with the back panel 11 against his or her back and front panel 12 against his or her abdomen, and then the other strap 13 or 14 can be pulled tight and brought into contact with the outside surface 12e of the front panel 12 in order to form a complete, closed loop around the wearer's torso to hold the back brace 10 in place on the wearer's torso.

Once the wearer is wearing the back brace as just described, the dynamic tensioning system 25 can now be used to easily adjust the brace 10 to provide the desired level of compressive force on the wearer's torso in order to provide the desired compression on the wearer's back. In the illustrated embodiment, the dynamic tensioning system 25 comprises two essentially identical strap assemblies 15, 16. Each strap assembly comprises at least an elastic webbing. In the particular embodiment illustrated in the drawings, each strap assembly comprises a non-elastic webbing (or strap) 30 and two elastic (preferably, semi-elastic) webbing segments 31, 32. As used herein, the term semi-elastic refers to the property of stretching in response to increasing force over a certain range and then becoming inelastic upon reaching a predetermined force.

The two semi-elastic webbings 31, 32 are sewn together at an oblique angle to each other to form a V-shape. They are further sewn to the first end 30a of the non-elastic webbing 30. Thus, the entire strap assembly 15 or 16 essentially has a Y-shape. In other embodiments, the two elastic strap segments 31, 32 may be replaced by a single strap segment formed in a V-shape. In another embodiment, a single piece of elastic webbing can be folded into a V-shape and passed through a square ring attached to the end of the inelastic webbing 30.

Even further, the V-shape itself is merely exemplary. Hence, in other embodiments, the entire strap assembly 15 or 16 may be a straight strap comprising two strap portions attached end-to-end, one strap portion being inelastic and the other strap portion being elastic.

Even further, the entire strap assembly 15 or 16 may be formed of an elastic strap without any inelastic strap portion.

In any event, returning to the particular illustrated embodiment, the non-elastic webbing 30 includes one of the hook and loop surface 34 of a hook and loop attaching system at least near the second end 30b of the webbing 30 for purposes of enabling removable attachment of the second end 30b of the strap assembly 15, 16 to the front panel 12 of the back brace 10, as will be described in more detail further below.

As can be seen in the drawings, the dynamic tension strap assemblies 15, 16 are permanently attached to the semi-rigid plastic piece 17 of the back panel 11. This is a desirable feature in some cases because it reduces the possibility of losing the dynamic tension strap assemblies 15, 16. Attachment may be achieved in any reasonable fashion. In the illustrated embodiment, the semi-elastic webbings 31, 32 are folded over onto themselves at their ends 31 b, 32b and sewn together in order to form loops 33 for accepting square rings 34, which, in turn, are attached to semi-rigid plastic piece 17 via metal plates 36 (with loops 36a for accepting square rings 34), and rivets 37, as shown. The illustrated design allows the strap assemblies to pivot about the axis of the rivet which helps prevent the webbings from bunching up or folding onto themselves. In other embodiments, the dynamic tension strap assemblies 15, 16 may be directly attached to the back panel 11 by any other reasonable means, such as rivets, screws, adhesive, snaps, buttons, buckles, hook and loop systems, etc.

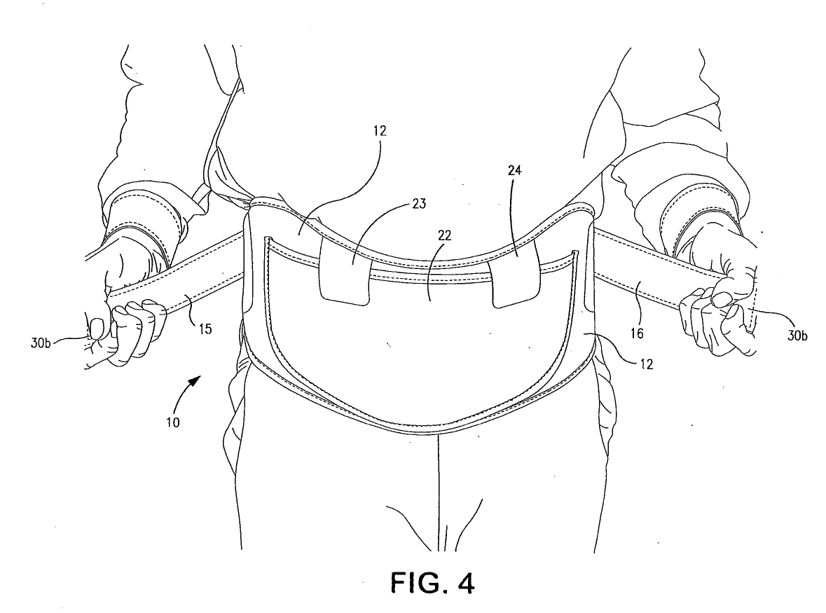

When the wearer is wearing the brace 10 as previously described, whereby it is wrapped around the wearer's torso and the two side straps 13, 14 are attached to both the front and back panels 11, 12 to form a complete loop around the wearer's torso, the wearer (or a third person, such as a physician or physician's assistant) can grab one of the dynamic tension strap assemblies 15, 16 in each hand, as shown in FIG. 4, and pull them forward to pull the back panel 11 tighter to the wearer's back (i.e., compress the wearer's back). When the desired tension is reached, the ends 30b of the dynamic tension straps 15, 16 may be attached to the front panel 12 of the brace 10 to maintain the compression force after the wearer (or third person) lets go of the dynamic tension strap assemblies 15, 16. In this particular embodiment using a hook and loop attachment system, the wearer merely needs to press the second ends 30b of the dynamic tension strap assemblies 15, 16 against the front panel 12 and they will be attached thereto by means of the hook and loop attaching system. The hook and loop attaching system allows easy attachment and detachment for readjusting the compression force and/or taking the back brace 10 on and off.

The use of mating hook and loop surfaces on the strap assemblies 15, 16 and outside surface 12e of the front panel 12 is particularly useful because it is both easy to attach and detach and is essentially infinitely adjustable. However, there are any number of other ways to provide adjustable attachment of the dynamic tension strap assemblies 15, 16 to the front panel. For instance, each strap assembly 15, 16 may be provided with both the hook and the loop surface on different parts of the strap assembly and the front panel may have a square ring or other loop that the end 30b of the strap assembly can be passed through and then the strap assembly may be folded over on itself to attach the hook and loop portions to each other. Additional possibilities include providing buckles on the front panel for adjustably locking the straps at any desired tension.

In one embodiment of the invention, the elastic webbing portions 31, 32 of the dynamic tension strap assemblies 15, 16 are semi-elastic and reach maximum stretch at 200 pounds of force.

In one embodiment, the stretchable span of the semi-elastic straps (the portion that is between connection points to the brace and that is free to stretch) is about 4 inches long when untensioned and about 6.5 inches when under maximum elastic stretch.

When the dynamic tension system 25 is deployed as described above, it may relieve some or all of the tension in the side straps 13, 14. Hence, the dynamic tension strap assemblies 15, 16 may primarily or exclusively provide the compression force on the wearer's back. The compression provided by the elasticity of the dynamic tension strap assemblies 15, 16 provides dynamic tension on the wearer's back, providing several advantages. For instance, it allows the back brace 10 to adapt in accordance with twisting and other motions of the wearer without restricting the wearer's movement and without causing the back panel 11 to lose contact with the wearer's back.

The back brace of the present invention is simple to measure and fit to wearers of widely varied ages and sizes. However, it also may be provided in different sizes in order to better accommodate different sized wearers. In particular, the size and/or shape of the back panel 11 and/or the front panel 12 can be altered to accommodate different sized wearers. In addition, the back brace can be offered in different sizes for hyperextending different portions of the spine, including standard LSO, full LSO, and TLSO sizes.

Furthermore, skin breakdown is reduced due to the slot 18 in the back piece 17. The brace is easy to use and is light in weight, providing consistent compression and pain relief.

The brace 10 can be used post-operatively and the semi-rigid back piece 17 may be made heat moldable in order to achieve a more custom fit, if necessary.

The ambulatory wearer may begin a conservative exercise program, thus allowing the wearer to strengthen the supportive muscular system, thereby promoting independence and a return to normal everyday activities.

Having thus described a few particular embodiments of the invention, various alterations, modifications, and improvements will readily occur to those skilled in the art. Such alterations, modifications and improvements as are made obvious by this disclosure are intended to be part of this description though not expressly stated herein, and are intended to be within the spirit and scope of the invention. Accordingly, the foregoing description is by way of example only, and not limiting. The invention is limited only as defined in the following claims and equivalents thereto.

1. A back brace comprising:
a back panel for placement against a wearer's back;
a front panel for placement against a wearer's abdomen; and
at least one dynamic tension strap assembly for connecting the front panel to the back panel so as to compress the wearer's torso between the front and back panels.

2. The back brace of claim 1 wherein the at least one dynamic tension strap assembly comprises first and second dynamic tension strap assemblies, extending in substantially opposite lateral direction from the back panel.

3. The back brace of claim 2 wherein the first and second dynamic tensioning strap assemblies are permanently attached to one of the front panel and the back panel and are removably attachable to the other of the front panel and the back panel.

4. The back brace of claim 2 wherein the first and second dynamic tensioning strap assemblies are pivotally attached to one of the front panel and the back panel.

5. The back brace of claim 3 further comprising a hook and loop attaching system for removably attaching the first and second strap assemblies to the front panel.

6. The back brace of claim 2 wherein the back panel includes a slot position to correspond in position with the spine of the wearer of the back brace.

7. The back brace of claim 2 further comprising first and second side straps for further attaching the front panel to the back panel.

8. The back brace of claim 2 wherein each of the first and second dynamic tension strap assemblies comprises an inelastic webbing attached to an elastic webbing.

9. The back brace of claim 8 wherein each dynamic tension strap assembly comprises a first end comprising the inelastic webbing for attaching to the front panel and a second end comprising the elastic webbing for attaching to the back panel.

10 The back brace of claim 9 wherein the elastic webbing comprises first and second elastic webbing segments disposed at an oblique angle to each other to form a V-shape, and wherein a first longitudinal end of the first elastic webbing segment is attached to the back panel at a first point and a first longitudinal end of the second elastic webbing segment is attached to the back panel at a second point and a second longitudinal end of the first elastic webbing segment, a second longitudinal end of the second elastic webbing segment, and a first longitudinal end of the inelastic webbing segment are attached to each other.

11. The back brace of claim 10 wherein the first ends of the first and second elastic webbing segments are pivotally attached to the back panel.

12. The back brace of claim 8 wherein the elastic webbing is semi-elastic.

13. The back brace of claim 12 wherein the elastic webbing becomes inelastic at 200 pounds of force.

14. A back brace comprising:
a back panel including a semi-rigid piece and padding, having a top side, a bottom side, a left lateral side, and a right lateral side;
a front panel including padding and having a left lateral side, and a right lateral side;
a first side strap extending between the left lateral side of the back panel and the left lateral side of the front panel;
a second side strap extending between the right lateral side of the back panel and the right lateral side of the front panel;
a first dynamic tension strap assembly having a first end attached to the left lateral side of the back panel, the first dynamic tension strap assembly including a semi-elastic webbing;
a second dynamic tension strap assembly having a first end attached to the right lateral side of the back panel, the second dynamic tension strap assembly including a semi-elastic webbing;
each of the first and second dynamic tension strap assemblies having a second end adapted to be removably attached to the front panel under tension.

15. The back brace of claim 14 wherein the first and second dynamic tension strap assemblies each include at least one of a hook surface and loop surface of a hook and loop attaching system for attaching the second end of the dynamic tension strap assemblies to the front panel.

16. The back brace of claim 15 wherein the second ends of the first and second dynamic tension strap assemblies include one of a hook surface and a loop surface of a hook and loop attachment system and the front panel includes the other of the hook surface and loop surface of the hook and loop attachment system.

17. The back brace of claim 14 wherein each of the first and second dynamic tension strap assemblies comprises an inelastic webbing attached to an elastic webbing.

18. The back brace of claim 17 wherein each dynamic tension strap assembly comprises a first end for attaching to the front panel comprising the inelastic webbing and a second end for attaching to the back panel comprising the elastic webbing.

19. The back brace of claim 18 wherein the elastic webbing comprises first and second elastic webbing segments disposed at an oblique angle to each other to form a V-shape, and wherein a first longitudinal end of the first elastic webbing segment is attached to the back panel at a first point and a first longitudinal end of the second elastic webbing segment is attached to the back panel at a second point and a second longitudinal end of the first elastic webbing segment, a second longitudinal end of the. second elastic webbing segment, and a first end of the first, inelastic webbing are attached to each other.

20. The back brace comprising:
a back panel comprising a semi-rigid piece;
a front panel;
at least one elastic strap for attaching the back panel and the front panel to each other in a loop around a wearer's torso; and
means for adjustably attaching the at least one elastic strap to one of the front panel and the back panel under different tensions.

21. The back brace of claim 20 wherein the means for adjustably attaching comprising a hook and loop attaching system.
